(19) United States
(12) Patent Application Publication
(10) Pub. No.: US 2015/0366723 A1
(43) Pub. Date: Dec. 24, 2015

(54) SECTIONED DIAPER

(71) Applicant: Shelley S. Bath, Niagara Falls, NY (US)

(72) Inventor: Shelley S. Bath, Niagara Falls, NY (US)

(21) Appl. No.: 14/311,193

(22) Filed: Jun. 20, 2014

(51) Int. Cl. A61F 13/49

(52) U.S. Cl. CPC A61F 13/49; A61F 2013/49098; A61F 2013/15048

(57) ABSTRACT

A sectioned diaper may include a diaper having an interior area, a first section, a second section, and a middle portion. The first section and the second section may be divided by the middle portion. The middle portion, in various embodiments, may prevent any discharge from the anus to come in contact with the vagina or penis, thereby reducing the possibility of urinary tract infections, renal infections, bacteremia and the like.

### BACKGROUND OF THE INVENTION

The present invention relates to diapers and, more particularly, to a sectioned diaper.

Currently, diapers allow stool to freely soil the genital area of an infant such as the vagina or penis, often leading to irritation to the vaginal area from excessive wiping and also exposed the area to infection, such as urinary tract infections. For male infants, urinary tract infections will also occur, especially when they are uncircumcised.

As can be seen, there is a need for a sectioned diaper that may decrease the chances of urinary tract infections, renal infections and bacteremia.

### SUMMARY OF THE INVENTION

In one aspect of the present invention, a sectioned diaper comprises: a diaper having an interior area, a first section, a second section, and a middle portion, wherein the first section and the second section are divided by the middle portion.

In another aspect of the present invention, a method for separating bodily function areas within a diaper comprises: placing a sectioned diaper on a surface with an interior area facing away from the surface, wherein the sectioned diaper is defined as a diaper having the interior area, a first section, a second section and a middle portion, wherein the first section and the second section are divided by the middle portion; positioning an infant on the sectioned diaper so that a genital area of the infant is positioned within the first section of the sectioned diaper and an anus of the infant is positioned within the second section of the sectioned diaper; and securing the sectioned diaper onto the infant.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is an exploded view of an exemplary embodiment of the present invention;

FIG. 3 is a perspective view of an alternate embodiment of the present invention;

FIG. 4 is a perspective view of an alternate embodiment of the present invention, laid flat;

FIG. 5 is a section view of an exemplary embodiment of the present invention, taken along line 5-5 in FIG. 3;

FIG. 6 is a perspective view of an alternate embodiment of the present invention;

FIG. 7 is a section view of an exemplary embodiment of the present invention, taken along line 7-7 in FIG. 6;

FIG. 8 is a perspective view of an alternate embodiment of the present invention, laid flat;

FIG. 9 is a perspective view of an alternate embodiment of the invention with a foldable flap in a folded down position;

FIG. 10 is a section view of an exemplary embodiment of the present invention, taken along line 10-10 in FIG. 9;

FIG. 11 is a perspective view of an alternate embodiment of the present invention, laid flat;

FIG. 12 is a perspective view of an alternate embodiment of the present invention, with a foldable cup in a folded down position; and

FIG. 13 is a section view of an exemplary embodiment of the present invention, taken along line 13-13 in FIG. 12.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a sectioned diaper that may include a diaper having an interior area, a first section, a second section, and a middle portion. The first section and the second section may be divided by the middle portion. The middle portion, in various embodiments, may prevent any discharge from the anus to come in contact with the vagina or penis, thereby reducing the possibility of urinary tract infections, renal infections, bacteremia and the like.

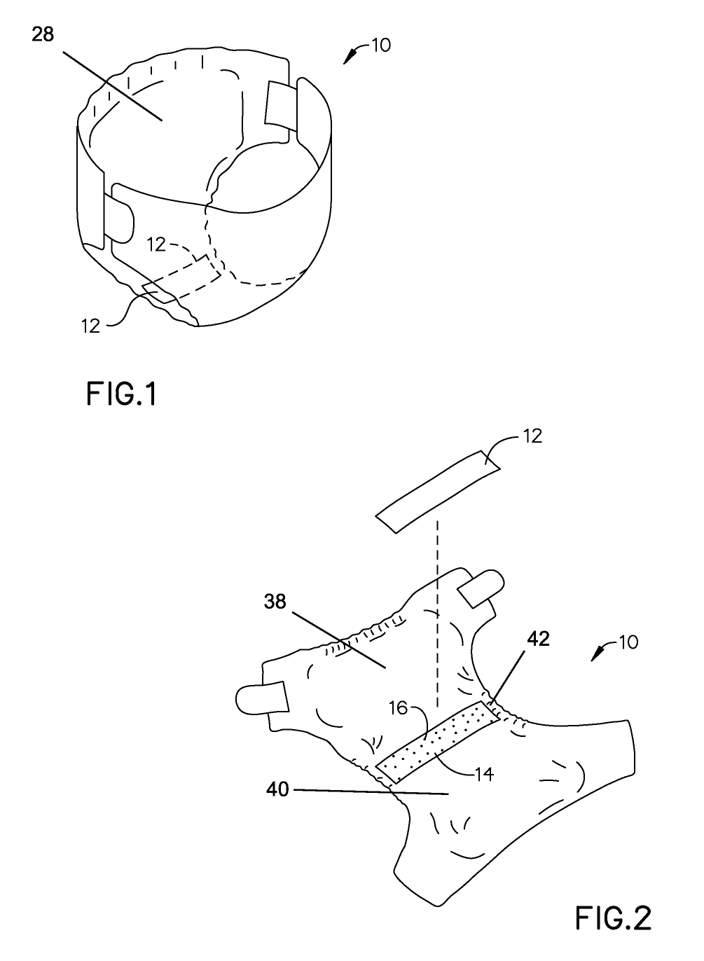

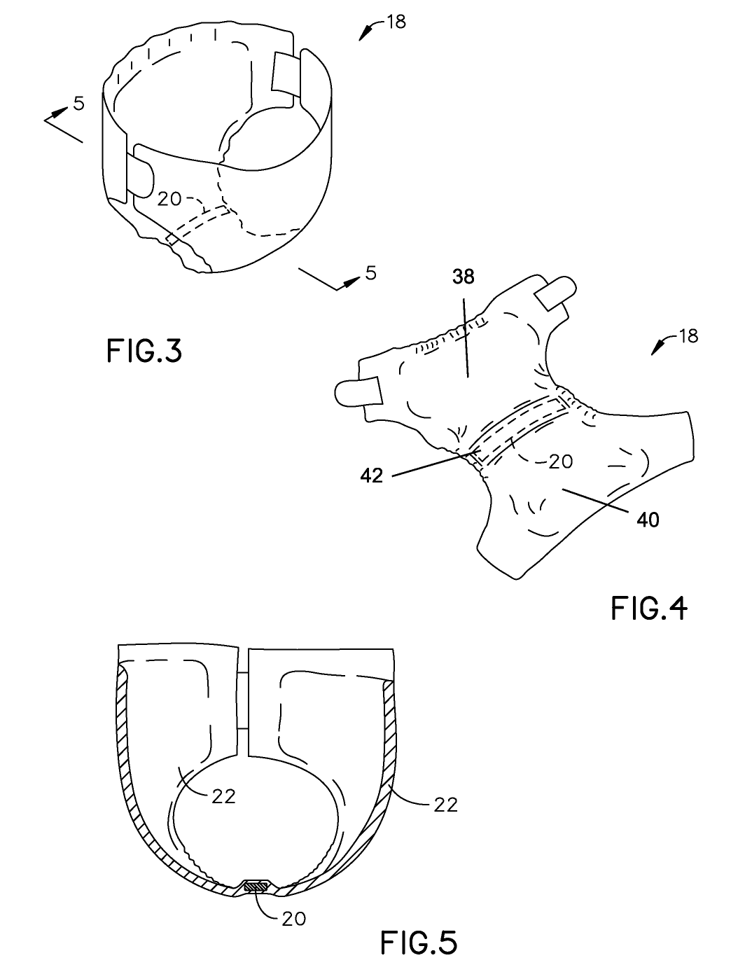

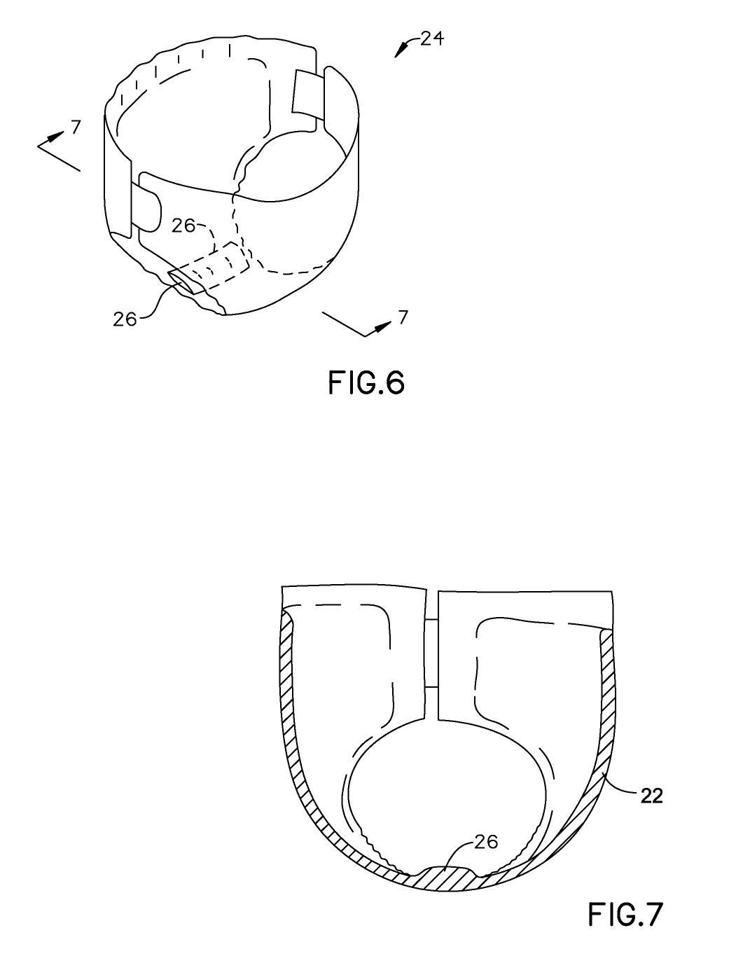

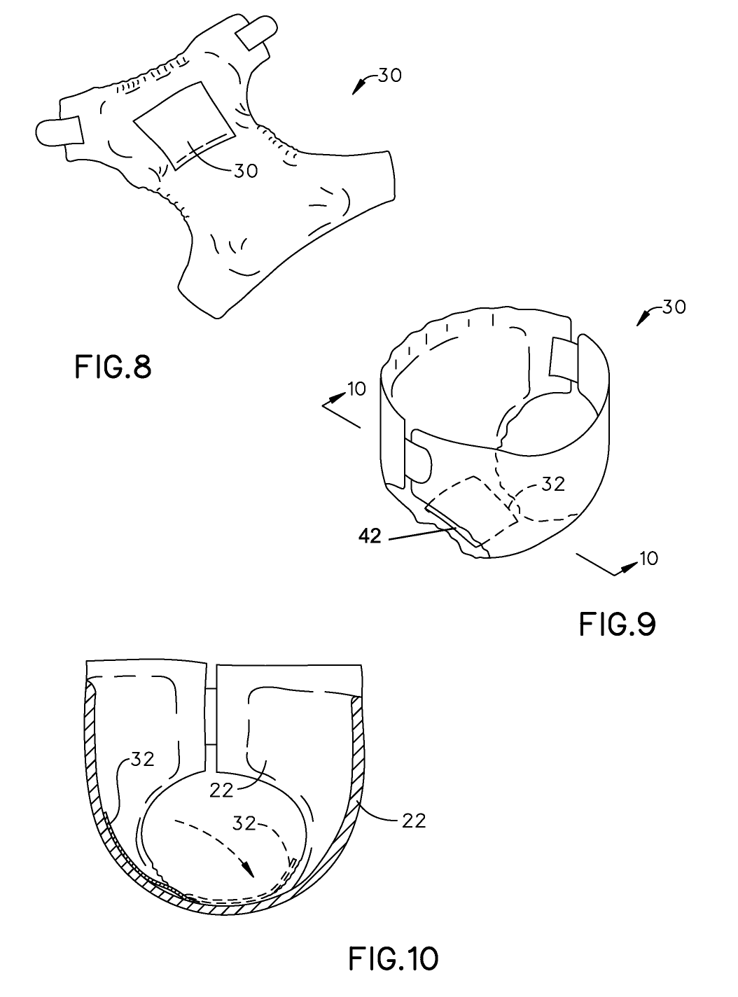

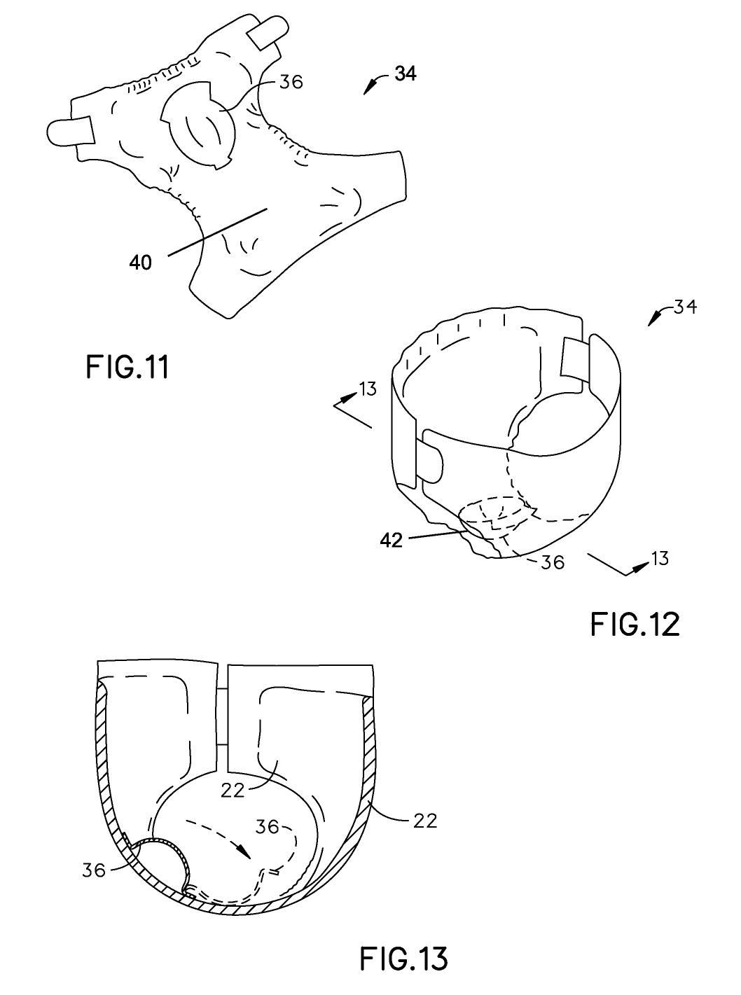

As is illustrated in FIGS. 1 through 7, a sectioned diaper may include a diaper having an interior area 28, a first section 38, a second section 40 and a middle portion 42. The first section 38 and the second section 40 may be divided by the middle portion 42.

In certain embodiments, the middle portion 42 of an adhesive embodiment 10 may include an adhesive strip 14. An adhesive strip cover 12 may be placed over the adhesive strip 14 until the sectioned diaper may be in use. The adhesive strip 14 may be made from a skin sensitive material 16. In certain embodiments, the middle portion 42 of a strip embodiment 18 may include a strip 20. The strip 20 may be a moldable strip. The strip 20 may be located within a diaper lining 22 and may run across the middle portion 42 between a first side to a second side. In certain embodiments, the middle portion 42 of a built up region embodiment 24 may include a built up region 26 that is raised above the rest of the interior area 28. In certain embodiments, the middle portion 42 of an embodiment may include a foldable component. The foldable component may be a foldable flap 32 in a foldable flap embodiment 30, a foldable cup 36 in a foldable cup embodiment 34, or the like. In the embodiments with the foldable component, the foldable component may initially be in the first section 38 of the sectioned diaper or in the second section 40 of the sectioned diaper. The foldable component may fold down and mold around the vagina/penis or may fold down and stick in between the vagina/penis and the anus to create a barrier between a genital area of the wearer and the anus area of the wearer.

A method of using the sectioned diaper may include the following. A person may place the sectioned diaper on a surface. The interior area 28 may be facing away from the surface. A person, such as an infant, or the like, may be positioned on the sectioned diaper. The genital area of the infant may be positioned over the first section 38 of the sectioned diaper. For female infants, the genital area may include a vagina. For male infants, the genital area may include a penis. The anus of the infant may be positioned over the second section 40 of the sectioned diaper. The middle portion 42 may be positioned in between the genital area and the anus, dividing the first section 38 and the second section 40. The middle portion 42 provides a barrier between the first section 38 and the second section 40. The sectioned diaper may then be secured onto the infant. The sectioned diaper may be secured by tape or the like. The middle portion 42 may prevent or reduce any discharge from the anus to come in contact with the vagina or penis, thereby reducing the possibility of urinary tract infections, renal infections, bacteremia and the like.

It should be understood, of course, that the foregoing relates to exemplary embodiments of the invention and that modifications may be made without departing from the spirit and scope of the invention as set forth in the following claims.

1. A sectioned diaper comprising:
a diaper having an interior area, a first section, a second section, and a middle portion, wherein the middle portion provides a barrier between the first section and the second section; and
a foldable component pivotably connected to the middle portion so as to be movable from a first position to a second position, wherein the second position is adapted to operatively engage a genital area of a person positioned on the interior area.

2. The sectioned diaper of claim 1, wherein the middle portion comprises an adhesive strip.

3. The sectioned diaper of claim 2, further comprising an adhesive strip cover removably attached to the adhesive strip.

4. The sectioned diaper of claim 1, wherein the middle portion comprises a moldable strip within an interior housing of a diaper lining.

5. The sectioned diaper of claim 1, wherein the middle portion comprises a built up region that is raised above the rest of the interior area.

6. The sectioned diaper of claim 1, wherein the foldable component is a flap.

7. A method for separating bodily function areas within a diaper comprising:
placing a sectioned diaper on a surface with an interior area facing away from the surface, wherein the sectioned diaper comprises a diaper having the interior area, a first section, a second section and a middle portion, wherein the middle portion provides a barrier between the first section and the second section, and wherein a foldable component is pivotably connected to the middle portion so as to be movable from a first position to a second position;
positioning a person on the sectioned diaper so that a genital area of the person is positioned within the first section of the sectioned diaper and an anus of the infant is positioned within the second section of the sectioned diaper;
pivoting the foldable component to the second position so that the foldable component operatively engages the genital area of a person; and
securing the sectioned diaper onto the person.

8. The method of claim 7, wherein the middle portion of the sectioned diaper comprises an adhesive strip.

9. The method of claim 8, wherein the middle portion of the sectioned diaper further comprising an adhesive strip cover removably attached to the adhesive strip.

10. The method of claim 7, wherein the middle portion of the sectioned diaper comprises a moldable strip within an interior housing of a diaper lining.

11. The method of claim 7, wherein the middle portion of the sectioned diaper comprises a built up region that is raised above the rest of the interior area.

12. (canceled)

13. The sectioned diaper of claim 1, wherein the foldable component in the first position is disposed along a portion of the interior area of the middle portion.

14. The sectioned diaper of claim 1, wherein the foldable component in the second position is not disposed along a portion of the interior area of the middle portion.

15. The sectioned diaper of claim 1, wherein the foldable component is cup-shaped.

16. The sectioned diaper of claim 1, wherein the foldable component in the second position is adapted to be molded around the genital area of the person positioned on the interior area.

17. The sectioned diaper of claim 1, wherein the foldable component in the second position is adapted to stick between the genital area and an anus of the person positioned on the interior area.

18. The method of claim 7, further comprising molding the foldable component in the second position around the genital area of the person.

19. The method of claim 7, further comprising sticking the foldable component in the second position between the genital area and the anus of the person.
